(19) United States
(12) Patent Application Publication
(10) Pub. No.: US 2012/0123349 A1
(43) Pub. Date: May 17, 2012

(54) SYRINGE SHARP TIP GUARD

(76) Inventor: Thomas Chun, Northridge, CA (US)

(21) Appl. No.: 12/946,089

(22) Filed: Nov. 15, 2010

(51) Int. Cl. A61M 5/32

(52) U.S. Cl. 604/198

(57) ABSTRACT

A guard for the sharp tip of a needle of an injection syringe. The guard may comprise two components which are separate from and readily attachable to the syringe. One component is stationary and the other movable relative to the syringe. The movable member moves by expanding longitudinally to cover the otherwise exposed needle tip when the plunger of the syringe is depressed beyond a predetermined degree. The stationary member releasably holds the movable member in the initial position, and releases the movable member to expand and thus deploy when the predetermined degree of depression is imposed by the user of the syringe.

### FIELD OF THE INVENTION

The present invention relates to syringes such as medical transdermal syringes, and more particularly to a guard which may be installed over most conventional syringes. The guard expands to cover the exposed needle tip after an injection is completed.

### BACKGROUND OF THE INVENTION

Syringes for delivering injections into the body have long been known. Such syringes become hazardous after use if no steps are taken to protect medical personnel and others from the exposed sharp tip. Instances of inadvertent transmission of infectious diseases due to accidental pricking from a used needle have become a known hazard in the medical field.

Various steps to counter this hazard have been proposed. One solution is to place a rubber or cork member over the exposed tip. Another solution is to modify syringes to include integral deployable shields which project on demand to cover the otherwise exposed sharp tip. These and other approaches all have various drawbacks. Placing a rubber member over an exposed sharp tip must be performed immediately upon completion of an injection, requires the medical personnel to maintain the rubber member at the ready for installation, and requires one additional manual step in its installation. It would be possible for the installer to slip and cause the very inadvertent prick that is intended to be avoided.

Integral deployable shields entail complication and additional costs. Beyond these objections, there exists a substantial supply of conventional syringes which have not been provided with such protection. It would be desirable to impart the protection of integral deployable shields to the existing supply of conventional syringes.

### SUMMARY OF THE INVENTION

The present invention provides an uncomplicated, almost automatically operating needle guard for covering the otherwise exposed sharp tips of needles of syringes. The guard may comprise two parts which may be installed concentrically over a conventional syringe. The first one of the two parts remains stationary relative to the barrel of the syringe. The second part automatically extends dynamically and longitudinally from the first part, so as to cover the exposed needle of the syringe, responsive to the plunger of the syringe being depressed beyond that degree necessary to discharge the contents of the syringe. No specific action by the medical personnel is required to deploy the protective feature beyond removing the syringe from the patient and continuing to depress the plunger of the syringe.

In one implementation of the invention, the extensible second part has integral spring characteristics which provide motive force for dynamically extending the second part, which increases its length from the initial length. The first part restrains the second part from extending until the appropriate time. Continued pressure exerted by the user and applied to the syringe is transferred to the extensible second part to cause the second part to deploy.

The two parts may be inexpensively and advantageously fabricated from suitable synthetic resins by ordinary fabrication methods, such as injection molding. These two parts may be dimensioned and configured to cooperate with conventional syringe designs, thereby enabling the novel feature to work with existing syringes which were not originally adapted to provide protective covering of the sharp needle tips.

It is an advantage of the invention that an effective needle guard may be formed from only two separate components. Also, each of these two components may be molded from an ordinary synthetic resin. Further, a spring which imparts propulsion to move the guard element into the deployed position may be integral with either one of the two components, and may further be formed from the same constituent material of its associated component. No separate spring need be provided, and the spring need not be for example a metallic coil spring.

It is an object of the invention to provide readily performed, almost automatic protection for the otherwise sharp exposed tip of a syringe after the syringe has been used to deliver an injection.

Another object of the invention is to provide a protective arrangement which is readily used with existing or conventional syringe designs.

A further object is to require as few as two components to provide the novel protective arrangement.

Still another object of the invention is to be able to limit constituent materials of any one or both of the two components to an ordinary synthetic resin.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is an exploded, partly cross sectional side view of the novel needle tip guard arrangement, shown proximate a conventional syringe.

FIG. 2 is a side view of two of the components of FIG. 1 shown assembled in operable relationship to one another, and with the syringe shown at the top of FIG. 1 omitted.

FIG. 3 corresponds to FIG. 2, but illustrates an expanded or extended condition of one of the components of FIG. 2.

FIG. 4A shows the components of FIG. 1 assembled in operable relationship, prior to depression of the plunger of the syringe, as it would be in preparation for an injection.

FIG. 4B is similar to FIG. 4A, but shows a subsequent condition wherein the plunger of the syringe has been slightly depressed.

FIG. 4C is similar to FIG. 4B, but shows a further degree of depression of the plunger.

FIG. 4D shows the final stage in the progression of FIGS. 4A-4D, wherein a needle cover has been deployed by progressive depressing of the plunger, and also shows how the assembly may be gripped in the hand of a person.

FIG. 5 is an enlarged detail view corresponding to FIG. 4D, shown partly in cross section.

FIG. 6 is an enlarged perspective detail view of a portion of the length of the center component of FIG. 1, shown as it would appear if spread open by pulling from both ends by finger pressure.

FIG. 7 is an enlarged side view of an alternative to the structure of FIG. 6

### DETAILED DESCRIPTION

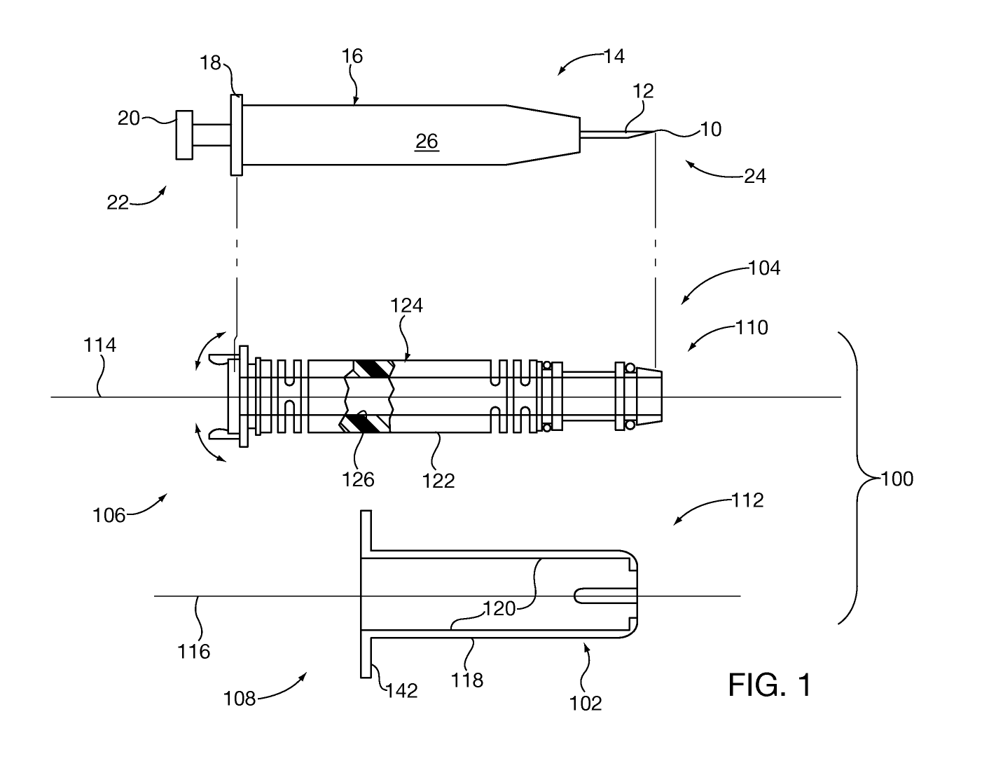

Referring first to FIG. 1, according to at least one aspect of the invention, there is shown a guard arrangement 100 for covering the sharp tip 10 of a needle 12 of a syringe 14. The syringe 14 may be conventional, having a housing 16 bearing an outwardly extending flange 18 and a movable needle assembly which comprises a plunger 20 and the needle 12.

The guard arrangement 100 may comprise two separable and complementing components including a stationary member 102 which engages the housing 16 of the syringe 14, and a mobile guard member 104. When assembled together for use, the stationary member 102 and the guard member 104 are coupled to one another in a manner enabling the mobile guard member 104 to move between an initial position wherein the sharp tip 10 of the needle 12 is exposed and a deployed position wherein the sharp tip 10 of the needle 12 is covered so that casual inadvertent pricking is substantially prevented.

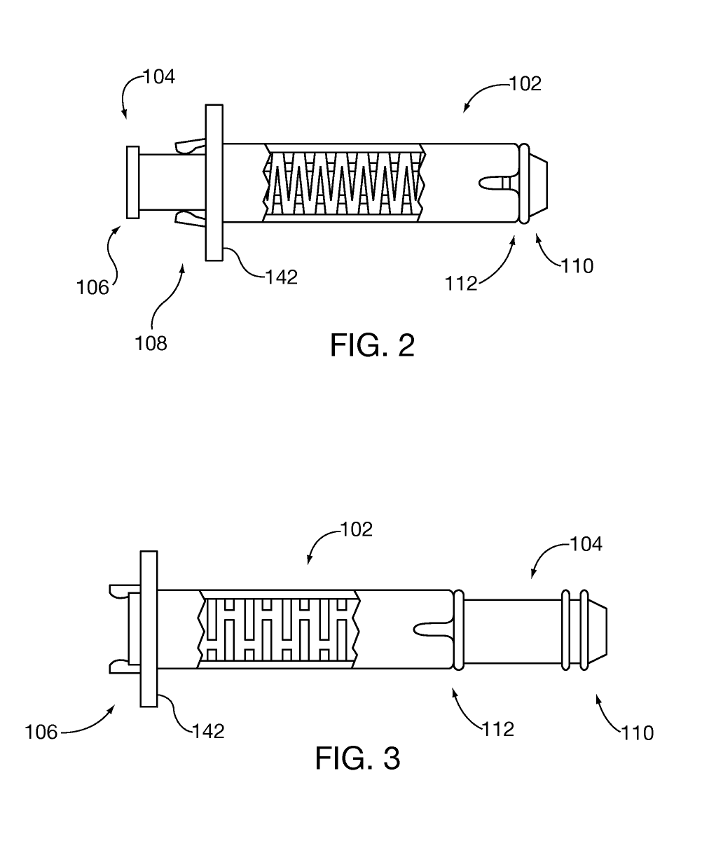

FIG. 2 shows the stationary member 102 coupled to the guard member 104 as they would be in the initial position. In FIG. 2, the syringe 14 seen in FIG. 1 would be inserted concentrically within the mobile guard member 104 from the left. FIG. 3 shows the stationary member 102 coupled to the guard member 104 as they would be in the deployed position, again in the absence of the syringe 14.

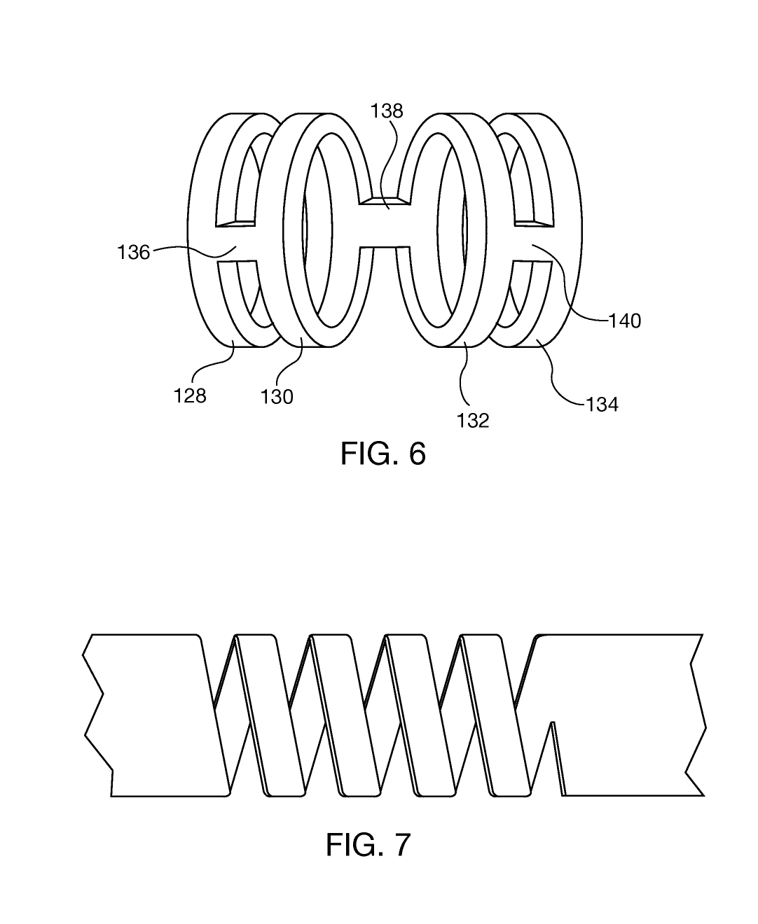

It will be seen that a spring arrangement formed in the wall of the guard member 104, which spring arrangement will be described further hereinafter with reference to FIG. 6, is in a compressed state in FIG. 2. Hence the overall length of the guard member 104 is less in FIG. 2 than in FIG. 3, where the spring arrangement is shown expanded or extended.

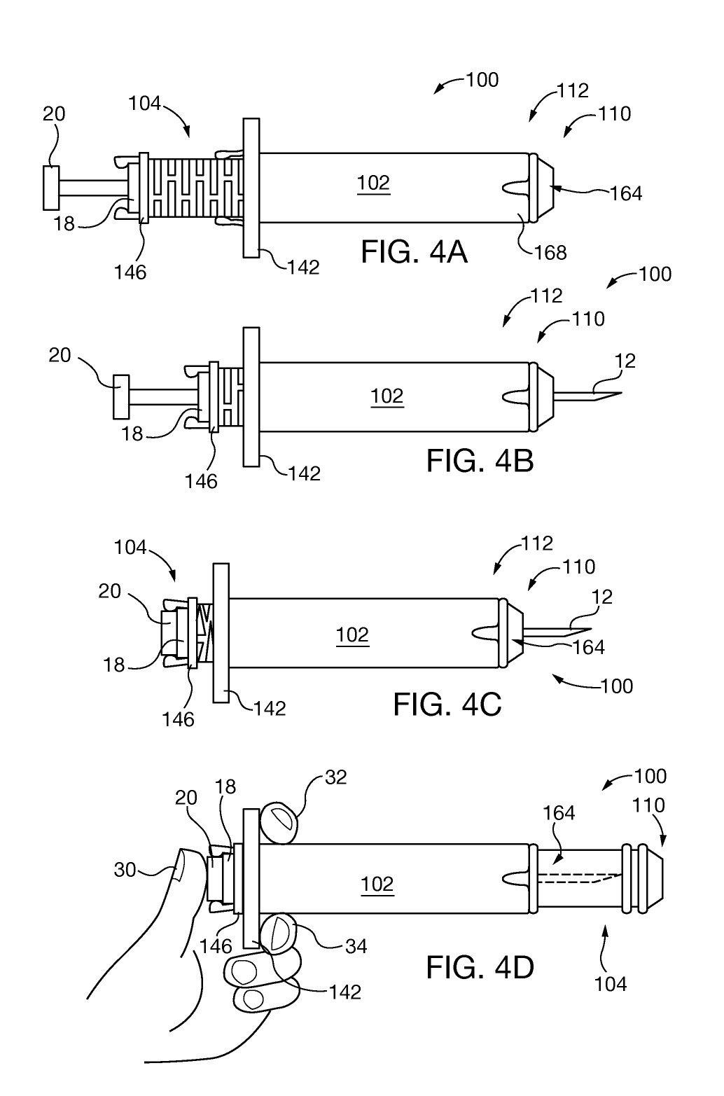

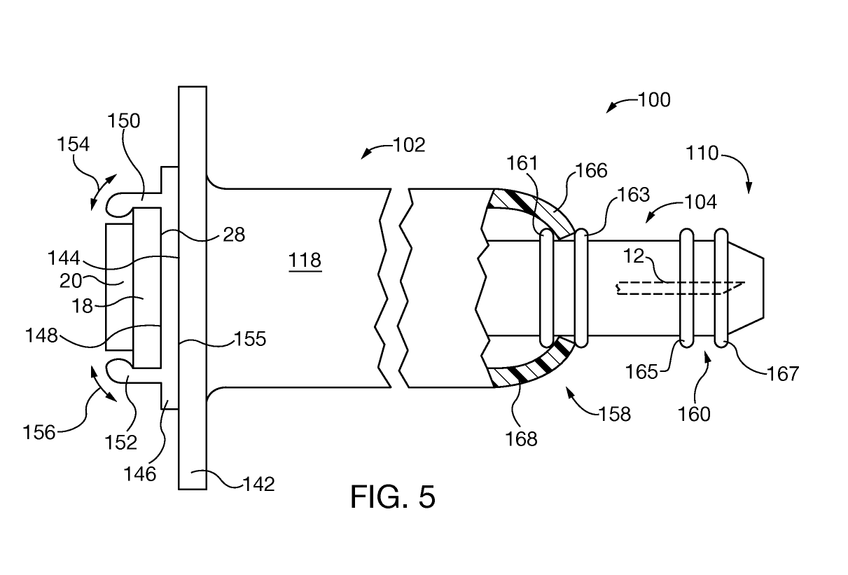

FIG. 4A corresponds to FIG. 2, showing the stationary member 102 coupled to the guard member 104 in the initial position, but also showing the syringe 14 installed. The plunger 20 projects from the proximal end 106 of the guard member 104. For purposes of semantic description, the plunger 20 will be understood to define the proximal end 22 of the syringe 14, the proximal end 106 of the guard member 104, and the proximal end 108 of the stationary member 102. Similarly, the needle 12 will be understood to define the distal end 24 of the syringe 14, the distal end 110 of the guard member 104, and the distal end 112 of the stationary member 102. Of course, these relationships exist when the syringe 14, the stationary member 102, and the guard member 104 are all mutually assembled in concentric relation as seen in FIGS. 4A, 4B, 4C, 4D and 5.

Concentricity signifies that the center line 114 of the guard member 104, the center line 116 of the stationary member 102, and the length of the needle 12 are aligned in common. Of course, it would be possible to vary configuration of the syringe 14, the guard member 104, and the stationary member 102 such that true concentricity among these components does not occur, while still retaining the functional aspects of the invention. Therefore, concentricity will be understood to be employed in an explanatory or rhetorical capacity, and not as a limitation. Alternatively stated, the configuration of the stationary member 102 and of the guard member 104 may each have a longitudinal dimension which coincides with the longitudinal dimension of the needle 12 of the syringe 14.

In FIGS. 4D and 5, the guard member 104 is disposed to be concentrically outside the housing 16 of the syringe 14 (FIG. 1). The stationary member 102 is in turn disposed to be concentrically outside the circumferential wall 122 of the guard member 104. In FIG. 4D, the plunger 20 is seen to be depressed to the point of contact with the flange 18, which in turn contacts the flange 146 of the guard member 104, which again in turn has come to abut the flange 142 of the stationary member 102.

Referring again to FIG. 4B, slight depression of the plunger 20 both causes the needle 12 to project visibly from the stationary member 102 in preparation for an injection. It should also be noted that the entire guard member 104 has moved slightly to the right responsive to depression of the plunger 20. The spring feature shown compressed in FIG. 2 and uncompressed or fully extended in FIG. 3 remains uncompressed or fully extended in FIG. 4B. Hence, the overall length of the guard member 104 remains at its full length as seen in FIG. 4B.

FIG. 4C shows the syringe 14 and the guard arrangement 100 just after the plunger 20 has fully discharged the contents of the syringe 14. The guard member 104 has become compressed and has been received within the stationary member 102. The needle 12 projects beyond the guard arrangement member 100. The overall length of the guard member 104 is at its minimum in the condition depicted in FIG. 4C.

In FIG. 4D, the plunger has been depressed not only so as to discharge contents of the syringe 14 for purposes of an injection, but slightly more, to the point that a predetermined threshold of force has been applied to the guard member 104. That force and slight degree of movement of the plunger 20 act to transfer force to the guard member 104 which in turn causes the guard member 104 to extend from the maximally compressed condition seen in FIG. 4C to an extended condition characterizing the deployed position seen in FIGS. 4D and 5. It will be seen by comparing FIG. 4A to FIG. 4D that projection of the distal end 110 of the guard member 104 has now caused the distal end 110 of the guard member 104 to cover the needle 12, thus all but eliminating the possibility of inadvertent pricking by the tip 10 of the needle 12.

The overall function of the guard arrangement 100 has now been explained. This function is dependent upon coupling of the distal end 112 of the stationary member 102 to the guard member 104. This coupling will be described further hereinafter.

Referring again to FIG. 1, the guard arrangement 100 may comprise a guide element for constraining the mobile guard member 104 to move only longitudinally along the housing 16 of the syringe 14. Of course, longitudinal movement will be understood to be movement in the direction of the length of the needle 12 and the center lines 114 and 116. The guide element may comprise a circumferential wall 118 which is part of the stationary member 102, and an internal surface 120 of the circumferential wall 118. The internal surface 120 may cooperate with the outer surface 122 of a circumferential wall 124 of the guard member 104. In turn, the circumferential wall 124 may have an internal circumferential surface 126 which is adapted to slidably cooperate with the outer circumferential surface 26 extending along the length of the housing 16 of the syringe 14. Thus the circumferential wall 124 of the guard member 104 may surround and slidably engage the circumferential surface 26 of the housing 16 of the syringe 14. In similar fashion, the circumferential wall 118 of the stationary member 102 may surround and slidably engage the circumferential wall 124 of the guard member 104.

The stationary member 102 and the guard member 104 may collectively provide an anchoring feature which opposes movement of the proximal end 106 of the guard member 104 relative to the housing 16 of the syringe 14. Alternatively stated, the proximal end 106 of the guard member 104 may be pinned by interference to the syringe 14, even as the distal end 110 of the guard member 104 continues to move longitudinally along the syringe 14 when moving to cover the needle 12 and its tip 10.

To achieve this result, the guard member 104 may comprise a self-contained length expansion feature which is biased to urge the guard member 104 to assume greater overall length when moving to the deployed position from the initial position. This self-contained expansion feature may comprise a spring arrangement which acts to urge the distal end 110 away from the proximal end 106. The self-contained expansion feature may be implemented in any one of several ways. For example, the guard member 104 may be fabricated from one and only one constituent substance, such as a resilient synthetic resin of known type, such as nylon or Delrin®. Spring forces of the spring arrangement may arise from inherent spring characteristics of the constituent substance, and may be enhanced by configuration of the guard member 104.

Referring to FIG. 6, the configuration of the guard member 104 may comprise a pattern of solid members and intervening voids extending in the longitudinal dimension, wherein the voids of the pattern can collapse and extend responsive to compression and extension of the guard member 104 in the longitudinal dimension. In the example of FIG. 6, the solid members may comprise a series of rings 128, 130, 132, 134 arranged parallel but spaced apart from adjacent rings 128, 130, 132, 134 by connector bars 136, 138, 140. It will be seen that the connector bars 136, 138, 140 are staggered in that each connector bar such as the connector bars 136, 138, 140 is angularly displaced from an adjacent connector bar by one hundred eighty degrees, where each ring such as the rings 128, 130, 132, 134 are circular and extend about a circle of three hundred sixty degrees. The resultant pattern of solid members (such as the rings 128, 130, 132, 134 and the connector bars 136, 138, 140) form intervening voids which extend only circumferentially about the pattern when no external compressive forces and no external tensile forces are imposed on the guard member 104.

Regardless of its exact configuration and nature, the pattern of solid members and voids of the guard member 104, together with the resilient nature of the selected constituent material, act to urge the guard member 104 into the expanded condition seen in FIG. 1. When the guard member 104 is assembled into the operable assembly comprising the syringe 14, the guard member 104, and the stationary member 102 as seen in FIG. 4, the guard member 104 has been manually compressed in the longitudinal dimension of the syringe 14. The guard member 104 is constrained against spontaneous expansion in the longitudinal dimension. The engagement element referred to prior, which may contribute to securing the stationary member 102 to the syringe 14, may comprise the following surfaces and associated members.

As seen in FIG. 5, the stationary member 102 may comprise an outwardly projecting flange 142 which may come to abut the flange 18 of the syringe 14. The flange 142 may have an abutment surface 144 which abuts an opposed abutment surface 28 of the flange 18. The flange 142 also receives the fingertips of a person when the person is administering an injection with the guard arrangement 100 installed on a syringe such as the syringe 14.

When the syringe 14 is assembled to the guard arrangement 100 and the user imposes pressure by thumb 30 against the plunger 20 while holding the guard arrangement 100 with two fingers 32 and 34, as seen in FIG. 4D, interference between the respective abutment surfaces 28 and 144 will act to cause the stationary member 102 to engage the syringe 14. Caught and compressed between the flange 18 and the fingers 32 and 34, the stationary member 102 will remain immobile or stationary relative to the housing 16 of the syringe 14.

The guard member 104 may comprise a flange 146, which after assembly as shown in FIG. 4 may be located between the flange 18 and the flange 142. The flange 146 may present an abutment surface 148 which comes to contact the abutment surface 28 of the flange 18 of the syringe 14. In turn, the abutment surface 144 of the flange 142 establishes interference with an abutment surface 155 of the flange 146.

As interferences are established by contact of the flanges 18, 142, and 146, it will be appreciated that such interferences anchor the proximal end 106 of the guard member 104 to the housing 16 of the syringe 14 when the plunger 20 is depressed. The abutment surfaces 28 and 148 establish the interference which prevents the proximal end 106 of the mobile guard member 104 from moving relative to the housing 16 of the syringe 14 when the plunger 20 of the syringe 14 is depressed.

Advantageously seen in the enlarged scale of FIG. 5 are resilient clips 150, 152 which releasably engage the flange 18 of the syringe 14. The clips 150, 152 may pivot as indicated by respective arrows 154, 156 when engaging and releasing the flange 18. Any number of such clips may be provided, as desired. The clips 150, 152 serve as an engagement element for engaging the housing 16 of the syringe 14.

Coupling of the distal end 112 of the stationary member 102 to the guard member 104 may be accomplished in the following way. The stationary member 102 and the guard member 104 may comprise a system of grooves formed in the guard member 104 which entrap jaws formed in the stationary member 102. The guard member 104 may comprise a first outwardly facing groove 158 and a second outwardly facing groove 160 which is longitudinally spaced apart from the first outwardly facing groove 158.

As shown, the grooves 158, 160 are defined between two opposed outwardly projecting ribs 161, 163, 165, 167. As an alternative, grooves such as the grooves 158, 160 could be depressed into the guard member 104. Also, grooves such as the grooves 158, 160 could be formed by a combination of depressions with raised ribs such as the ribs 161, 163, 165, 167.

The stationary member 102 may comprise a resilient inwardly facing projection which is dimensioned and configured to releasably seat selectively within the first outwardly facing groove 158 and the second outwardly facing groove 160. The resilient inwardly facing projection may be formed in segments. A slot 164 divides the distal end 112 of the circumferential wall 118 of the stationary member 102 into upper and lower sections or jaws 166, 168, as depicted in FIG. 5. It should be noted at this point that orientational terms such as upper and lower refer to the subject drawing as viewed by an observer. Therefore, orientational terms must be understood to provide semantic basis for purposes of description, and do not limit the invention or its component parts in any particular way.

Respective ends 170, 172 of the upper and lower jaws 166, 168 may be regarded respectively as inwardly facing projections. The ends 170, 172 come to be received and seated within either the first outwardly facing groove 158 or the second outwardly facing groove 160, depending on the degree of extension of the guard member 104 from the stationary member 102.

In the initial position illustrated for example in FIG. 4A, the upper jaw 166 and lower jaw 168 had been received and seated within the second outwardly facing groove 160. However, as force imposed on the plunger 20 is ultimately transferred to the guard member 104 by a suitable inference, resiliency of the jaws 166, 168 enable the jaws 166, 168 to spread apart, in turn enabling the distal end 110 of the guard member 104 to extend to the deployed position shown in FIG. 4D. The jaws 166, 168 then come to seat in the outwardly facing groove 158, which establishes interference opposing further extension of the distal end 110 of the guard member. In the deployed position shown in FIG. 4D and FIG. 5, the needle 12 is seen to be covered by the guard member 104.

A significant advantage of the invention is that only two physically separate components need be fabricated and assembled to provide benefits of the invention. Notably, a guard arrangement such as the guard arrangement 100 may consist of two and only two physically separate and mutually detachable and movable components, notably, the stationary member 102 and the guard member 104. As employed herein, the phrase “physically separate” signifies that the two components may be provided to the end user as two and only two components, although the two components may be preassembled to one another by the manufacturer, distributor, or other sales or distribution party if desired. The phrase “mutually detachable” signifies that the two components are manually detachable from one another without impairing their efficacy, and may be reassembled for capable of subsequent usages. The phrase “mutually movable” signifies that after assembly to a syringe such as the syringe 14, the two components may move relative to one another in the course of use and function as described.

It is not necessary that an entire component move when moving. As explained priorly, one or both of the components, such as either a stationary member such as the stationary member 102 or a guard member such as the guard member 104 may be constructed such that one part of it moves while another part of it remains stationary. An example of this is seen with the guard member 14, which is of variable length by virtue of the constituent material, configuration which accommodates stretching and other deformations, or both.

It is a further advantage that one or both of the two components of the novel guard arrangement 100 comprise a spring which is formed integrally therewith, which spring contributes to moving the guard member 104 to the deployed position. This enables the user to move the guard member 104 to the deployed position merely by continuing to depress the plunger 20 of the syringe 14. The spring feature described herein, also referred to as a self-contained length expansion feature, is for the purposes of this invention regarded as an integral spring. It should be further noted that the integrated spring may be formed from the same constituent material of its associated component, i.e., the guard member 104.

Of course, the spring feature may be fabricated as part of the stationary member 102. It would also be possible to have two springs, each integrated into one of the stationary member 102 and also the guard member 104 (these options are not illustrated).

It is a further advantage of the invention that a suitable spring need not be provided as a separate member which must be for example embedded into either the stationary member 102 or the guard member 104.

It will be appreciated that a novel guard arrangement such as the guard arrangement 100 comprises the stationary member 102, the mobile guard member 104, and a propulsion arrangement disposed to move the mobile guard member 104 from the initial position to the deployed position. The propulsion arrangement may comprise a spring such as the spring arrangement integrated into the mobile guard member 104, and furthermore, a non-metallic spring. The non-metallic spring may be integral with either the stationary member 102, the mobile guard member 104, or may comprise two springs, each formed integrally with one of the stationary member 102 and the mobile guard member 104.

The non-metallic spring may have spring characteristics arising from inherent elasticity of the constituent material of its associated component, such as the stationary member 102 or the mobile guard member 104. The constituent material may be a synthetic resin.

The non-metallic spring may comprise voids for accommodating compression and expansion of the non-metallic spring.

It will also be appreciated that the novel guard arrangement may be provided by a total of only two physically separate components, such as consisting of a first member such as the stationary member 102 and a second member such as the guard member 104.

Regardless of the number of components forming the novel guard arrangement, it will be appreciated that at least one of the stationary member and the mobile guard member comprises an integral propulsion arrangement comprising a non-metallic spring disposed to move the mobile guard member from the initial position to the deployed position.

It is still another advantage that a guard arrangement such as the guard arrangement 100 has a manually releasable latch which releasably secures the mobile guard member 104 in the deployed position. The coupling feature arising from the jaws 166 and 168 and their cooperation with the grooves 158 and 160 provide such a feature.

Still another advantage of the invention is that a guard arrangement such as the guard arrangement 100 wherein depressing the plunger of the syringe causes the needle of the syringe to move to an exposed position to enable the needle to pass through the skin of a patient, and continued depression of the plunger causes the mobile guard member 104 to move to the deployed position. Alternatively stated, the same manual action by the user which effects the injection also initiates and actuates deployment of the guard member 104.

The present invention is susceptible to modifications and variations which may be introduced thereto without departing from the inventive concepts. For example, cooperating components of the stationary member 102 and the guard member 104 may be interchanged, where such modification would not impair operability. Male and female components, such as the projections formed by the jaws 166 and 168, and the grooves 158 and 160 could be interchanged.

Monolithic construction of the guard member 104 could be provided in other ways. For example, a guard member (not shown) which in other ways is the functional equivalent of the guard member 104 could be formed to have a molded plastic proximal end, a molded plastic distal end, and a metallic coil spring embedded in the proximal and distal ends. A guard member (not shown) could be molded from dissimilar synthetic resins injected into a common mold, or otherwise fabricated from dissimilar materials including dissimilar metals, or a synthetic resin and a metal.

Where the spring feature which urges the guard member 104 to expand has solid members and voids, there may be only one solid member and one intervening void, as occurs in the helical arrangement shown in FIG. 7. Configuration of the spring portion of the spring feature may assume configurations other than those illustrated and described herein. The spring portion may include more than one configuration, such as those configurations shown in FIGS. 6 and 7, or still other configurations if desired.

The relative locations of the stationary and mobile members, such as the stationary member 102 and the guard member 104 may be reversed if desired.

A guard member such as the guard member 104 may be arranged such that the entire guard member moves to cover the needle, rather than expanding in overall length. Alternatively, a guard member such as the guard member 104 may be arranged such that the guard member both changes in overall length, either expanding or contracting longitudinally, and also moves in its entirety to cover the needle.

Where interferences exist, they may be either directly acting or indirectly acting. That is, a component that is intended to be moved or stopped by another component may be directly in contact, or alternatively, an intervening member which transfers forces may be present.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A guard arrangement for covering the sharp tip of a needle of a syringe having a housing bearing an outwardly extending flange and a movable needle assembly comprising a plunger and a needle having a sharp tip, comprising:
a stationary member which engages the housing of the syringe; and
a mobile guard member which is coupled to the stationary member in a manner enabling the mobile guard member to move between an initial position wherein the sharp tip of the needle is exposed and a deployed position wherein the sharp tip of the needle is covered, wherein the stationary member has a guide element for constraining the mobile guard member to move longitudinally along the housing of the syringe, wherein
the stationary member and the mobile guard member are coupled to one another so as to oppose movement of the mobile guard member relative to the needle of the syringe until a predetermined threshold of force is applied to the mobile guard member and causes the mobile guard member to extend from the initial position to the deployed position covering the needle when force applied longitudinally to the mobile guard member exceeds the predetermined threshold of force.

2. The guard arrangement of claim 1, wherein the mobile guard member comprises a self-contained length expansion feature which is biased to urge the mobile guard member to assume greater overall length when moving to the deployed position.

3. The guard arrangement of claim 2, wherein the self-contained expansion feature comprises a spring arrangement.

4. The guard arrangement of claim 3, wherein the mobile guard member is fabricated from one and only one constituent substance, and spring force of the spring arrangement arises from inherent spring characteristics of the one and only one constituent substance and from configuration of the mobile guard member.

5. The guard arrangement of claim 4, wherein the one and only one constituent substance is a synthetic resin.

6. The guard arrangement of claim 4, wherein the configuration of the mobile guard member has a longitudinal dimension which coincides with the longitudinal dimension of the needle of the syringe, and the mobile guard member includes a pattern of solid members and intervening voids extending in the longitudinal dimension, wherein the voids of the pattern can collapse and extend responsive to compression and extension of the mobile guard member in the longitudinal dimension.

7. The guard arrangement of claim 6, wherein the solid members are configured such that the intervening voids extend only circumferentially about the pattern when no external compressive forces and no external tensile forces are imposed on the mobile guard member.

8. The guard arrangement of claim 1, wherein the syringe comprises an outer circumferential surface extending along the length of the syringe, and the guide element of the guard arrangement comprises a circumferential wall formed as part of the mobile guard member, which said circumferential wall surrounds and engages the circumferential surface of the syringe.

9. The guard arrangement of claim 1, wherein the mobile guard member comprises a proximal end proximate the plunger of the syringe and a distal end proximate the needle tip of the syringe, and an abutment surface which establishes interference between the proximal end of the mobile guard member and the housing of the syringe, which said interference acts to prevent the proximal end of the mobile guard member from moving relative to the housing of the syringe when the plunger of the syringe is depressed.

10. The guard arrangement of claim 1, wherein the stationary member comprises a resilient clip which releasably engages the flange of the syringe.

11. The guard arrangement of claim 1, wherein
the mobile guard member comprises a first outwardly facing groove and a second outwardly facing groove each formed therein, wherein the first outwardly facing groove and the second outwardly facing groove are longitudinally spaced apart from one another, and
the stationary member comprises a resilient inwardly facing projection formed therein, and wherein the resilient inwardly facing projection of the stationary member is dimensioned and configured to releasably seat within the first outwardly facing groove and the second outwardly facing groove of the mobile guard member such that the first outwardly facing groove, the second outwardly facing groove, and the inwardly facing projection couple the stationary member and the mobile guard member to one another so as to oppose movement of the mobile guard member relative to the needle of the syringe

12. The guard arrangement of claim 11, wherein the resilient inwardly facing projection formed in the stationary member comprises a plurality of segments and an equal number of resilient jaws formed in the stationary member and each supporting at least one of the segments.

13. The guard arrangement of claim 1, wherein the stationary member comprises an outwardly projecting flange for receiving the fingertips of a person when the person is administering an injection with the guard arrangement installed on a syringe.

14. The guard arrangement of claim 1, wherein the mobile guard member is disposed to be concentrically outside the housing of the syringe and the stationary member is disposed to be concentrically outside the circumferential wall of the mobile guard member.

15. A guard arrangement for covering the sharp tip of a needle of a syringe having a housing bearing an outwardly extending flange and a movable needle assembly comprising a plunger and a needle having a sharp tip, consisting of two and only two physically separate and mutually detachable and movable components, wherein
one of the only two physically separate and mutually detachable and movable components is a stationary member which is coupled to the housing of the syringe; and
the other one of the only two physically separate and mutually detachable and movable components is a mobile guard member which is coupled to the stationary member in a manner enabling the mobile guard member to move between an initial position wherein the sharp tip of the needle is exposed and a deployed position wherein the sharp tip of the needle is covered.

16. The guard arrangement of claim 15, wherein the mobile guard member is constrained to move only longitudinally along the housing of the syringe.

17. The guard arrangement of claim 15, wherein the stationary member and the mobile guard member collectively provide a releasable check feature which opposes movement of the mobile guard member relative to the housing of the syringe until a predetermined threshold of force is applied to the mobile guard member, and wherein the releasable check feature is disposed to cause the mobile guard member to extend from the initial position to the deployed position when force applied longitudinally to the mobile guard member exceeds the predetermined threshold of force.

18. The guard arrangement of claim 15, further comprising a spring formed integrally with at least one of the stationary member and the mobile guard member, wherein the spring is disposed to urge the mobile guard member to move to the deployed position.

19. The guard arrangement of claim 15, further comprising a manually releasable latch which releasably secures the mobile guard member in the deployed position.

20. A guard arrangement for covering the sharp tip of a needle of a syringe having a housing bearing an outwardly extending flange and a movable needle assembly comprising a plunger and a needle having a sharp tip, comprising
a stationary member which is coupled to the housing of the syringe; and
a mobile guard member which is coupled to the stationary member in a manner enabling the mobile guard member to move between an initial position wherein the sharp tip of the needle is exposed and a deployed position wherein the sharp tip of the needle is covered, wherein depressing the plunger of the syringe causes the needle of the syringe to move to an exposed position to enable the needle to pass through the skin of a patient, and continued depression of the plunger causes the mobile guard member to move to the deployed position.

21. A guard arrangement for covering the sharp tip of a needle of a syringe having a housing bearing an outwardly extending flange and a movable needle assembly comprising a plunger and a needle having a sharp tip, comprising:
a stationary member which engages the housing of the syringe;
a mobile guard member which is coupled to the stationary member in a manner enabling the mobile guard member to move between an initial position wherein the sharp tip of the needle is exposed and a deployed position wherein the sharp tip of the needle is covered; and
a propulsion arrangement comprising a non-metallic spring disposed to move the mobile guard member from the initial position to the deployed position.

22. The guard arrangement of claim 21, wherein the stationary member and the mobile guard member are coupled to one another so as to oppose movement of the mobile guard member relative to the needle of the syringe until a predetermined threshold of force is applied to the mobile guard member and causes the mobile guard member to extend from the initial position to the deployed position covering the needle when force applied longitudinally to the mobile guard member exceeds the predetermined threshold of force.

23. The guard arrangement of claim 21, wherein the non-metallic spring is integral with the mobile guard member.

24. The guard arrangement of claim 23, wherein the non-metallic spring has spring characteristics arising from inherent elasticity of the constituent material of the mobile guard member.

25. The guard arrangement of claim 23, wherein the non-metallic spring comprises voids for accommodating compression of the non-metallic spring.

26. The guard arrangement of claim 21, wherein the mobile guard member is fabricated from a synthetic resin.

27. A guard arrangement for covering the sharp tip of a needle of a syringe having a housing bearing an outwardly extending flange and a movable needle assembly comprising a plunger and a needle having a sharp tip, consisting of:
a stationary member which engages the housing of the syringe; and
a mobile guard member which is coupled to the stationary member and is disposed to move between an initial position wherein the sharp tip of the needle is exposed and a deployed position wherein the sharp tip of the needle is covered.

28. The guard arrangement of claim 27, wherein the stationary member comprises a guide element for constraining the mobile guard member to move only longitudinally along the housing of the syringe.

29. The guard arrangement of claim 27, wherein the stationary member and the mobile guard member are coupled to one another so as to oppose movement of the mobile guard member relative to the needle of the syringe until a predetermined threshold of force is applied to the mobile guard member and causes the mobile guard member to extend from the initial position to the deployed position covering the needle when force applied longitudinally to the mobile guard member exceeds the predetermined threshold of force.

30. The guard arrangement of claim 27, wherein one of the stationary member and the mobile guard member comprises an integral propulsion arrangement comprising a non-metallic spring disposed to move the mobile guard member from the initial position to the deployed position.
